(19) United States
(12) Patent Application Publication
(10) Pub. No.: US 2002/0102617 A1
(43) Pub. Date: Aug. 1, 2002

(54) Protein microarrays

(76) Inventors: Gavin MacBeath, Arlington, MA (US); Stuart L. Schreiber, Boston, MA (US); Peter K. Sorger, Cambridge, MA (US); Michael H. Cardone, Boston, MA (US); John Newman, Cambridge, MA (US)

Correspondence Address: Timothy J. Oyer, c/o Wolf, Greenfield & Sacks, P.C., Federal Reserve Plaza, 600 Atlantic Avenue, Boston, MA, 02210-2211, US

(21) Appl. No.: 09/923,243

(22) Filed: Aug. 3, 2001

Related U.S. Application Data
(60) Provisional application No. 60/222,709, filed on Aug. 3, 2000.
(60) Provisional application No. 60/297,897, filed on Jun. 13, 2001.

(51) Int. Cl. G01N 33/53; G01N 33/542; C12M 1/34

(52) U.S. Cl. 435/7.9; 435/287.2

(57) ABSTRACT

Driven by the influx of data from genome sequencing projects, systematic efforts are now underway to construct defined sets of cloned genes for high throughput expression and purification of recombinant proteins. To facilitate the subsequent study of protein function, the present invention provides protein microarrays that are compatible with the demand for extremely low sample volume and the rapid, simultaneous processing of thousands of proteins, and methods of assaying these arrays. The proteins are covalently or non-covalently attached to the surface of a solid support and retain their ability to interact specifically with other proteins, polynucleotides, other biological macromolecules, or small molecules.

### RELATED APPLICATIONS

The present application claims priority to provisional applications, U.S. Ser. No. 60/222,709, filed Aug. 3, 2000, and U.S. Ser. No. 60/297,897, filed Jun. 13, 2001, each of which is incorporated herein by reference in its entirety.

### BACKGROUND

Now that the entire genomes of several organisms including Saccharomyces cerevisiae and Escherichia coli have been sequenced and sequencing of the human genome is near completion, much effort is still required to understand the function of all the sequenced genes and the proteins they encode. Proteomics or the large scale understanding of proteins is still in its infancy and is becoming increasingly important. Many new tools and techniques need to be developed in order to study the function of all the proteins of a cell, tissue, and eventually organism in the same efficient manner as that of DNA microarrays. The sequencing of the genomes of whole organisms is only the beginning. Another 50 to 100 years will be needed to fully understand all genes and proteins discovered in the many genome sequencing projects currently underway.

Historically, genome-wide screens for protein function have been carried out using random cDNA expression libraries. Most frequently, the libraries are prepared in phage vectors and the expressed proteins immobilized on a membrane using a plaque lift procedure. The proteins are then assayed in situ on the filter and clones of interest identified by returning to the phage plaques. While this method has been effective for a variety of applications, including the identification of proteins based on antibody recognition (Young et al. Science 222:778-782, 1983; incorporated herein by reference), the identification of proteins that bind to specific peptides (Sparks et al. Nat. Biotechnol. 14:741-744, 1996; incorporated herein by reference), the identification of substrates for protein kinases (Fukunaga et al. Embo. J 16:1921-1933, 1997; incorporated herein by reference), and the identification of the targets of small molecules (Tanaka et al. Mol. Pharmacol. 55:356-363, 1999; incorporated herein by reference), it nevertheless suffers from several limitations. Most clones in the library do not encode proteins in the correct reading frame, and most proteins are not full-length. Also, bacterial expression of eukaryotic genes frequently fails to yield correctly folded proteins that are functional, and products derived from abundant transcripts are over-represented. Moreover, since plaque lifts are not amenable to miniaturization on the micrometer scale, it is hard to imagine screening all or many of the proteins of an organism hundreds or thousands of times using this approach. For example, while this method has been used successfully to identify potential substrates of the protein kinase Erk1 (Fukunaga et al. Embo. J 16:1921-1933, 1997; incorporated herein by reference), it is difficult to imagine running the same screen repeatedly using hundreds of different kinases.

With the advent of whole genome sequencing and high throughput molecular biology, it is now possible to construct perfectly normalized and spatially segregated sets of cloned genes (Fodor et al. Science 251:767-773, 1991; Lockhart et al Nat. Biotechnol. 14:1675-1680, 1996; each of which is incorporated herein by reference). UniGene sets in the form of PCR products have been used extensively over the past decade to construct DNA microarrays for the study of transcriptional regulation (Schena et al. Science 270:467-470, 1995; incorporated herein by reference). Recently, spatially segregated clones in expression vectors have been used to study protein function in vivo using the yeast two-hybrid system to identify protein-protein interactions (Bartel et al. Nat. Genet. 12:72-77, 1996; Uetz et al. Nature 403:623-627, 2000; each of which is incorporated herein by reference) and in vitro using biochemical assays performed with pools of purified proteins to identify enzymatic activities (Martzen et al. Science 286:1153-1155, 1999; incorporated herein by reference).

However, there remains a need for a system of microarraying proteins that will preserve the proteins' functions and allow for high density arrays in much the same way that researchers have been able to array nucleic acids. The fragility of proteins unfortunately presents a major problem in microarraying proteins that must be overcome if microarrays of proteins are to be fully embraced by the scientific community.

### SUMMARY OF THE INVENTION

The present invention provides a system for microarraying and/or immobilizing proteins on a solid support and identifying proteins with desired properties. Preferably, the proteins are arrayed in such a manner as to preserve the function of the proteins or regain their functionality once arrayed on the solid support. One protein may be arrayed, or many different proteins may be arrayed using this system. The surface of the solid support may be arrayed resulting in discrete spots with attached protein, or the entire surface or a portion of the surface of the solid support may be evenly coated with a protein.

In one aspect, the present invention provides arrays of proteins on a solid support. The proteins are arrayed on the solid support so that one spot containing a particular protein is spatially segregated from other spots on the solid support. Preferably, the spots of protein are separated by such a distance as to prevent contamination of one spot with another spot. The proteins arrayed on the support may be one type of protein or many different types of proteins. Preferably, the identity of the protein can be determined by its position in the array. In certain embodiments, the proteins are immobilized on the solid support by covalent attachment (e.g. Schiff's base formation, amide or urea bond formation, silylation, disulfide linkage, Michael addition). The covalent attachment should preferably not substantially affect the structure, function, or activity of the protein (e.g., catalytic activity, ability to bind other proteins, ability to bind nucleic acids, ability to bind small molecules, 3-D structure, etc.). In certain other embodiments, the proteins are immobilized on the solid support through non-covalent interactions. Certain arrays of special interest include those that are useful for high-throughput screening. The density of these arrays of special interest are, in certain embodiments, at least 1000 spots per cm², and in certain other embodiments at least 1500 spots per cm².

In another aspect, the present invention provides a solid support that is uniformly coated with a protein or mixture of proteins. In certain embodiments, protein-coated solid supports such as this may be used by subsequently spotting assay reagents onto the coated solid support, thereby resulting in discrete (i.e., spatially segregated) areas in which the coated protein is assayed. A different reagent may be spotted at each discrete area of the array resulting in many different assays of the coated protein.

In another aspect, the invention provides a method of preparing arrays of proteins. In certain embodiments, the preparation of the microarrays is carried out by a commercially available automated arrayer. The solid support is first treated in order to create chemically reactive groups on the surface of the support. These chemically reactive groups serve as sites to which the proteins can bind. In certain embodiments, glass slides are treated with an aldehyde-containing silane reagent. In the case of arraying smaller proteins, the solid support is activated by first attaching BSA to the surface and then activating the BSA to obtain functionalized sites on the BSA protein. The smaller proteins are then attached to the functionalized sites on the BSA protein. The proteins to be arrayed are, in certain embodiments, provided in substantially pure form in solutions. These solutions can be provided in nanoliter-scale volumes ranging from about 1 nL to about 1000 nL, in certain embodiments about 1 nL, and the protein solutions are delivered to the slide, yielding spots approximately 150-200 μm in diameter. In certain embodiments of special interest, the proteins to be arrayed are provided in a buffered aqueous solution containing a humectant (e.g., glycerol, polyethylene glycol) to prevent evaporation of the nanodroplets. The proteins should remain hydrated throughout the preparation, storage, and assaying of the array to prevent denaturation of the protein. The proteins are then contacted with the solid support facilitating attachment through the chemically active sites on the support. The sites on the array which do not contain arrayed protein may be blocked using another protein or small molecule. In certain embodiments, BSA, caseine, nonfat milk, glycine, or ethanolamine is used to block the microarray. To give but one example, the blocking of aldehyde slides with BSA serves not only to quench the unreacted groups on the support, but also to form a molecular layer of BSA that reduces nonspecific binding of other proteins to the surface in subsequent steps. In certain embodiments of special interest, the immobilized and arrayed proteins are functional and retain a substantial fraction of their original activity. The preparation of solid supports uniformly coated with a protein may be prepared using any techniques known in the art of coating.

In yet another aspect, the present invention provides a method of identifying proteins with desired properties. These properties may include a catalytic activity, an ability to bind another protein, an ability to bind a nucleic acid or small molecule, a substrate for phosphorylation, etc. An array of functional proteins is contacted with a biological macromolecule or small molecule of interest, and binding or a chemical reaction is detected in order to identify proteins with the desired property. These assays are preferably performed using commonly available reagents (e.g., monoclonal antibodies, recombinant proteins) and equipment (e.g., scanners used in analyzing DNA microarrays). The binding or other chemical reaction may be detected by any means known in the art including fluorescence, autoradiography, colorimetric, immunoassay, surface plasmon resonance, mass spectrometry, surface-enhanced raman spectroscopy, conductivity, elipsometry, etc. In certain embodiments, the detection is performed by an automated scanner with a resolution high enough to discriminate between the spatially segregated spots of the array.

In yet another aspect, the present invention provides a method of identifying molecules capable of disrupting a interaction between a chemical compound and a known ligand of the chemical compound. A slide is coated with a chemical compound, and the slide with the attached chemical compound is contacted with a ligand known to bind the chemical compound. Molecules to be screened for their ability to compete for binding with the ligand to the chemical compound are then incubated with the slide with the chemical compound/ligand complex. The ligand and/or the molecules to be screened may be arrayed on the slide in discrete spots. The chemical compound, ligand, and molecules to be screened may be peptides, proteins, polynucleotides, small molecules, carbohydrates, or lipids. In certain embodiments, the ligand and/or molecule to be screened is labeled with a fluorophore, and the loss of ligand is detected by the loss or gain of fluorescence.

### Definitions

The term array refers to an arrangement of entities in a pattern on a substrate. Although the pattern is typically a two-dimensional pattern, the pattern may also be a three-dimensional pattern. In a preferred embodiment, the position of an entity (e.g., protein) within the array can be used to determine the identity of the entity.

The term antibody refers to an immunoglobulin or parts thereof, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are preferred in the present invention.

A biological macromolecule is a polynucleotide (e.g., RNA, DNA, RNA/DNA hybrid), protein, peptide, lipid, natural product, or polysaccharide. The biological macromolecule may be naturally occurring or non-naturally occurring. In a preferred embodiment, a biological macromolecule has a molecular weight greater than 500 g/mol.

A ligand refers to any chemical compound, polynucleotide, peptide, protein, lipid, carbohydrate, small molecule, natural product, polymer, etc. that has a binding affinity for a target (e.g., a protein, carbohydrate, lipid, peptide, macromolecules, biological macromolecules, oligonucleotide, polynucleotide). Preferably, the target is a protein. In some embodiments, the ligand is specific for its target. In some embodiments, the ligand has a binding affinity for the target in the range of 100 mM to 1 pM, preferably 1 mM to 1 pM, more preferably 1 μM to 1 pM. The ligand may bind to its target via any means including hydrophobic interactions, hydrogen bonding, electrostatic interactions, van der Waals interactions, pi stacking, covalent bonding, magnetic interactions, etc.

Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolopyrimidine, 3-methyl adenosine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

A protein comprises a polymer of amino acid residues linked together by peptide (amide) bonds. The term, as used herein, refers to proteins, polypeptides, and peptides of any size, structure, or function. Typically, a protein will be at least three amino acids long, preferably at least 10 amino acids in length, more preferably at least 25 amino acids in length, and most preferably at least 50 amino acids in length. Proteins may also be greater than 100 amino acids in length. A protein may refer to an individual protein or a collection of proteins. A protein may refer to a full-length protein or a fragment of a protein. Inventive proteins preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a myristoyl group, a fatty acid group, a linker for conjugation, functionalization, or other modification, etc. A protein may also be a single molecule or may be a multi-molecular complex comprising proteins, lipids, RNA, DNA, carbohydrates, etc. A protein may be a natural or unnatural fragment of a naturally occurring protein or peptide. A protein may be naturally occurring, recombinant, or synthetic, or any combination of these.

The term small molecule, as used herein, refers to a non-peptidic, non-oligomeric organic compound either synthesized in the laboratory or found in nature. Small molecules, as used herein, can refer to compounds that are “natural product-like”, such as small molecule that are similar in structure to a natural product or are similar with respect to density of stereocenters, density of functional groups, ring systems, 3-D structure, etc.; however, the term “small molecule” is not limited to “natural product-like” compounds and may include compounds that are not based on and are not similar to known natural products. Rather, a small molecule is typically characterized in that it contains several carbon-carbon bonds, and has a molecular weight of less than 1500, although this characterization is not intended to be limiting for the purposes of the present invention. Examples of small molecules that occur in nature include, but are not limited to, taxol, dynemicin, and rapamycin. Examples of small molecules that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference) and pending application Ser. No. 09/121,922, entitled “Synthesis of Combinatorial Libraries of Compounds Reminiscent of Natural Products”, the entire contents of which are incorporated herein by reference.

The term solid support refers to any material which can be functionalized to attached proteins to either directly or indirectly. The solid support may be any shape (e.g., circular, flat, square, rectangular, spherical, cuboid) or size. The solid support may comprise a single material (e.g., glass) or multiple layers of material (e.g., metal coated with a monolayer of small molecules, glass coated with a BSA). The solid support may be made of glass, plastic, polymers, metals, ceramics, alloys, composites, etc.

The term substantial fraction refers to the percentage of a protein's activity remaining after attachment to the solid support in forming the protein microarray. For example, in the case of enzymatic activity, a substantial fraction of the enzyme's activity is in certain embodiments greater than 10-50%; in certain other embodiments greater than 75%; and in still other embodiments greater than 95%, 97%, 98%, 99%, or 99.9%. The activity may also be a protein's ability to bind another protein, a polynucleotide, or a small molecule. Preferably, the attached protein is able to bind its ligand or target at least 10-50% as well as the unattached protein, more preferably at least 75%, and most preferable at least 95%.

### BRIEF DESCRIPTION OF THE DRAWING

FIG. 1 shows the detection of protein-protein interactions on a glass slide. (A) Slide probed with 0.5 μg/ml BODIPY-FL-IgG. (B) Slide probed with 0.1 μg/ml Cy3-IκBα. (C) Slide probed with 0.5 μg/ml Cy5-FKBP12+100 nM rapamycin. (D) Slide probed with 0.5 μg/ml Cy5-FKBP12 (no rapamycin). (E) Slide probed with 0.5 μg/ml BODIPY-FL-IgG +0.1 g/ml Cy3-IκBα+0.5 μg/ml Cy5-FKBP12+100 nM rapamycin. In all panels, BODIPY-FL, Cy3, and Cy5 fluorescence were false-colored blue, green, and red, respectively.

FIG. 2 shows a protein microarray with 10,800 spots on a single slide. Protein G was printed 10,799 times. A single spot of GST-FRB was printed in row 27, column 109. The slide was probed with 0.5 μg/ml BODIPY-FL-IgG +0.5 μg/ml Cy5-FKBP12+100 nM rapamycin. BODIPY-FL and Cy5 fluorescence were false-colored blue and red, respectively.

FIG. 3 shows the detection of a substrate of a protein kinase on a glass slide. (A) Slide incubated with the catalytic subunit of cAMP-dependent protein kinase (PKA). (B) Slide incubated with casein kinase II (CKII). (C) Slide incubated with p42 MAP kinase (Erk1).

FIG. 4 shows compounds used for the identification of the targets of small molecules. All compounds were coupled to bovine serum albumin through their carboxylate groups (either directly or via a flexible linker).

FIG. 5 shows the detection of protein targets of small molecules using protein microarray printed on glass slides. (A) Slide probed with 10 μg/ml Alexa₄₈₈-BSA-1. (B) Slide probed with 10 μg/ml Cy5-BSA-2. (C) Slide probed with 10 μg/ml Cy3-BSA-3a. (D) Slide probed with 10 μg/ml Alexa488-BSA-1+10 μg/ml Cy5-BSA-2+10 μg/ml Cy3-BSA-3a. In all panels, BODIPY-FL, Cy3, and Cy5 fluorescences were false-colored blue, green, and red, respectively.

FIG. 6 shows how fluorescence intensity scales linearly with the concentration of solution-phase protein over four orders of magnitude. FRB was spotted on aldehyde slides in triplicate at a concentration of 1 mg/ml. The slides were then probed with Cy5FKBP12, ranging in concentration from 150 pg/ml to 20 μg/ml. All solutions contained 1 μM rapamycin.

FIG. 7 shows the detection of protein targets of low-affinity ligands using protein microarrays printed on glass slides. (A) Slide probed with Cy5-BSA-2+Cy3BSA-3a. (B) Slide probed with Cy5-BSA-2+Cy3-BSA-3b. (C) Slide probed with Cy5BSA-2+Cy3-BSA-3c. All conjugates were used at a concentration of 10 μg/ml. In all panels, Cy3 and Cy5 fluorescence were false-colored green and red, respectively.

FIG. 8 is a schematic illustration of the procedure used in screening for competitors of a protein-ligand interaction.

In 8b, aldehyde or BSA-NHS slides are coated with the target. The protein slide is then incubated with a known ligand which has been labeled for easy detection (8c). Molecules to be screened are then spotted on the slides to identify molecule that can compete with the ligand for binding to the target (8d).

In 8e, the slides are then scanned to detect any loss of labeled ligand.

FIG. 9 shows data for slides with C37, DCC 1, and DCC2 as the labeled ligand and “5-helix” of HIV gp120 as the target immobilized on the slide. All peptide were present at a concentration of 1 μM in 60% PBS/40% glycerol.

### DETAILED DESCRIPTION OF THE INVENTION

In recognition of the need for a system of microarraying proteins that will preserve the proteins' functions and allow for the preparation of high density arrays, the present invention provides novel protein arrays, systems of preparing microarrays of proteins, and methods for assaying the arrays. In certain embodiments of special interest, the proteins attached to the solid support of the microarray retain a substantial fraction of their activity when compared to that of the unattached proteins (i.e., the proteins are functional). Additionally, in certain other embodiments of special interest, the microarrays are prepared at a density of at least 1000 spots per cm² to allow for high throughput screening of proteins, and in still other embodiments, at least 1500 spots per cm². In still other embodiments, the solid supports are prepared with a relatively even coating of protein over the surface of the solid support. It will be appreciated that the solid support to which the proteins are attached may be any material suitable for functionalization to allow attachment of the proteins. Exemplary solid supports include, but are not limited to, glass, plastics, polymers, metal surfaces, and self-assembled monolayers. As will be discussed in more detail below, the attachment of the proteins to the solid support is accomplished through covalent (e.g., Schiff's base linkage, acylation) or non-covalent linkages (e.g., hydrogen bonding, hydrophobic interactions).

Certain exemplary embodiments are described in more detail below; however, it will be appreciated by those of skill in this art that these examples are not intended to limit the scope of the present invention.

Arraying Proteins on Glass Slides.

To construct protein microarrays, it is desirable, as in the case of DNA microarrays, to immobilize the protein samples on a solid support. In order to study the function of proteins, however, this must be done in a way that substantially preserves the folded conformation of the proteins. While other methods for arraying proteins have been reported (Bussow et al. Nucleic Acids Res. 26:5007-5008, 1998; Lueking et al. Anal. Biochem. 270:103-111, 1999; Mendoza et al. Biotechniques 27:778-780, 782-786, 788, 1999; each of which is incorporated herein by reference), only one other group has described the immobilization of proteins in a way that preserves their function (Arenkov et al. Anal. Biochem. 278:123-131, 2000; incorporated herein by reference). They use microfabricated polyacrylamide gel pads to capture proteins and then accelerate diffusion through the matrix by microelectrophoresis. One of the drawbacks to this approach is that the preparation of these protein microarrays requires specialized equipment and does not rely on the standard arrayers and scanners used in preparing and studying DNA microarrays. The use of gel pads also requires that the reagents being used to probe the array be readily diffusable through the gel's matrix. For reagents such as large proteins, the gel's matrix will impede the reaction of the reagent with the immobilized proteins of the array.

In contrast to the non-functional protein arrays and/or the limited functional protein arrays described above, the present invention approaches the immobilization of functional proteins by covalently or non-covalently attaching the proteins to the surface of the solid support (e.g., glass, polymer, metal, self-assembled monolayer, etc.). One of the primary objectives in pursuing this approach was to make this technology easily accessible and compatible with the standard instrumentation employed in the fabrication of DNA microarrays, a task which, heretofore, had not been accomplished.

In general, the method of preparing the protein arrays involves providing a solid support whose surface has been activated for the attachment of proteins and arraying the proteins onto the activated surface and allowing them to attach to the solid support directly or indirectly. Solid supports useful in the present invention include any material, including but not limited to, glass, plastics, polymers, metal, and self-assembled monolayers. The support may be provided in any shape or size. In certain embodiments, the solid support is a glass slide used in light microscopy. In certain embodiments, the surface of the solid support is modified with a chemical reagent to provide sites of attachment for the protein. The attachment may be through a covalent or non-covalent interaction.

The proteins may be obtained by any means known including chemical synthesis, solid phase synthesis, purification from natural sources, and purification from recombinant sources. In certain embodiments, the proteins are provided in substantially pure form. The protein may be greater than 75% pure, greater than 90% pure, or greater than 95% pure. In certain embodiment, the proteins are provided in aqueous solution. The proteins may be delivered to the surface of the activated solid support using any technique. This includes manual as well as automated techniques. In certain embodiments, an automated arrayer used for high-throughput screening is used to array the protein solutions onto the solid support.

A variety of chemically derivatized glass slides that can be printed on and imaged using commercially available arrayers and scanners may be used as a solid support for the microarrays. In certain embodiments, glass slides that have been treated with an aldehyde-containing silane reagent are used. In one embodiment of special interest, glass slides with aldehyde moieties attached are purchased from TeleChem International (Cupertino, California) under the trade name “SuperAldehyde Substrates”. The aldehyde groups on the surface of these slides react readily with primary amines on the proteins to form a Schiff's base linkage. Since typical proteins display many lysine residues on their surface, as well as the generally more reactive α-amine at their N-terminus, they can attach to the slide in a variety of orientations, permitting different sides of the protein to interact with other proteins, small molecules, or small molecules in solution.

It will be appreciated that specific linkages used in the present invention should be selected to be (1) robust enough so that the proteins are not inadvertently cleaved during subsequent assaying steps, and (2) inert so that the functionalities employed do not interfere with the subsequent assaying steps. The specific linkages to be used should also lead to microarrays of proteins with a substantial fraction of their original activity intact. In particular, the attached proteins should be functional with respect to the activity being assayed for. The specific chemistry used to attach the proteins to the solid support should also preferably be compatible with aqueous solutions such as those typically used in preparing, handling, and storing proteins.

Other covalent attachments may be used in the present invention including silylation and Michael addition reactions (described in U.S. Ser. No. 09/567,910, filed May 10, 2000; incorporated herein by reference). Other exemplary linkages include disulfide bonds, amide bonds, urea bonds, ester bonds, ether bonds, hydrazone linkages, and carbon-carbon bonds. The functional groups of the amino acids on the outer surface of the protein may be used in forming these linkages. For example, a cysteine residue with its thiol group may be used to form a disulfide bond or a thioether linkage, a lysine residue with its amino group may be used to form an amide bond or urea bond, glutamic acid or aspartic acid residues with their carboxylic acid groups may be used to form amide, carbonate, or ester bonds, or threonine and serine residues with their hydroxyl groups may be used to form ester or ether linkages.

In certain embodiment of special interest, the solid support is functionalized with maleimide groups to allow for the attachment of proteins through a Michael addition reaction (March, Advanced Organic Chemistry (4^{th} ed.), New York: John Wiley & Sons, 1992, 795-797; incorporated herein by reference). Proteins tagged with cysteine groups or with naturally occurring cysteine groups on the surface can then readily attach to the surface upon printing via the expected thioether linkage. Other electrophilic Michael acceptors (i.e., unsaturated carbon-carbon bonds in conjugation with at least one electron-withdrawing group) may be utilized in functionalizing the surface of the solid support; however, maleimides and vinyl sulfones are of special interest.

As discussed above, in certain other embodiments, the linkage of the protein to the solid support is accomplished through non-covalent interactions, such as van der Waals interactions, hydrogen bonding, hydrophobic interactions, pi stacking, etc. Examples of these non-covalent interactions include biotin-streptavidin, metal complex formation (e.g., histidine tag-nickel complex), nucleic acid hybridization, antibody-antigen interactions, etc. In certain embodiments, a recombinant protein is tagged with an epitope tag, and the solid support is activated by attaching anti-epitope antibodies to the solid support. In yet another particularly preferred embodiment, a recombinant protein is tagged with a poly-histidine tag, and the solid support comprises a metal ion surface (e.g., Ni⁺²) to which the poly-histidine tag can bind. As would be appreciated by one of ordinary skill in this art, the epitope/anti-epitope and the poly-histidine tag/metal cation approaches could be taken advantage of in purifying the protein in situ on the solid support. Contaminating proteins found in cell lysates that do not contain the epitope tag or the poly-histidine tag would not attach to solid support, and therefore, they could be easily washed away leaving only the desired tagged protein behind.

In still another embodiment involving non-covalent interactions, the interactions linking the protein to the solid support may be non-specific. In one embodiment, the arrayed proteins are attached to a hydrophobic surface (e.g., plastic) through hydrophobic interactions. In yet another embodiment, the protein is immobilized in a polymeric matrix. Examples of polymeric matrices useful in the present invention include, but are not limited to, polyacrylamide, dextran, hydrogel, and polysaccharides. In certain embodiments, the thickness of the polymeric matrix is less than 1 micron; and in still other embodiments less than 500 nm; and in yet other embodiments less than 100 nm.

In yet another embodiment, a polymeric matrix is covalently attached to the solid support and chemically activated, and the arrayed proteins are linked to the polymeric matrix by reacting with the chemically activated groups. To give but one example, polydextran is covalently attached to the slide and carboxymethylated. The carboxyl groups are then activated with EDC/NHS, or disuccinimidyl carbonate to form activated NHS esters. These chemically reactive ester groups can react with primary amines on the arrayed proteins to form amide linkages.

After the desired linkage is selected and the appropriate reagents (e.g., solid support, proteins) are provided, the method of the present invention involves the printing of these protein arrays. The printing of the protein onto the array may be done manually using pipetman, syringes, capillary tubes, or multi-channel pipetman, or may be performed by a machine or robot such as those known in the art of high-throughput screening. In but one example, to fabricate protein microarrays, a high precision, contact-printing robot (e.g., GMS 417 Arrayer (Affymetrix, Santa Clara, Calif.), or split pin arrayer constructed following directions on P. Brown's web page (http://cmgm.stanford.edu/pbrown; incorporated herein by reference)) is used to deliver nanoliter-scale volumes of protein samples to the slides, yielding spots approximately 150-200 μm in diameter (1600 spots per square centimeter). The proteins are printed in a buffered aqueous solution (e.g., phosphate-buffered saline (PBS), Tris-HCL, HEPES) with 40% glycerol included to prevent evaporation of the nanodroplets. In other embodiments, humectants or polymers (e.g., polyethylene glycol, glycerin, maltitol, polydextrose, sorbitol, cetyl alcohol, fatty alcohols, propylene glycol) other than glycerol may be used to prevent evaporation. As would be appreciated by one of skill in this art, it is important that the proteins remain hydrated throughout this and subsequent steps to prevent denaturation and/or loss of functionality. In another embodiment, the proteins are provided in organic solvents (e.g., DMSO, DMF) or in partially aqueous solutions (e.g., 10% DMSO in water).

Following an incubation to allow for attachment of the proteins to the solid support, the slides are optionally immersed in a buffer containing a blocking agent such as bovine serum albumin (BSA). This serves not only to quench the unreacted aldehyde groups on the slide, but also to form a molecular layer of the blocking agent that reduces nonspecific binding of other proteins to the surface in subsequent steps. Other blocking agents include small molecules (e.g., glycine, ethanolamine) and other proteins (e.g., caseine, nonfat milk). In a certain embodiments, when peptides or very small proteins are printed, the blocking agent utilized is a small molecule (e.g. glycine, ethanolamine, ethylenediamine) so as not to obscure the proteins of interest.

In another aspect of the present invention, when peptides or very small molecules, such as those less than 5,500 Da) are printed, protein arrays are constructed utilizing a solid support having a molecular monolayer of BSA attached thereto. In certain embodiments, BSA-NHS slides are utilized and are fabricated by first attaching a molecular monolayer of BSA to the surface of glass slides and then activating the surface of the BSA with N,N′-disuccinimidyl carbonate. Alternatively, an aldehyde slide can be used to print a protein as small as 5,550 kD. Can be determined empirically by one of skill in this art. The activated lysine, aspartate, and glutamate residues on the BSA react readily with surface amines on the printed proteins to form covalent urea or amide linkages. The slides are subsequently quenched with glycine. In contrast to the aldehyde slides, proteins or peptides printed on BSA-coated slides are displayed on top of the BSA monolayer, rendering them accessible to macromolecules in solution. It will be appreciated that other readily available and easily functionalized proteins (e.g., caseine) or macromolecules (e.g., dextran) may be used in place of the BSA, and can be functionalized to allow for attachment of a protein of interest. It will be appreciated that the protein or macromolecule should not substantially interfere with the desired property or assay to be used. In certain embodiment a well-characterized and easily available protein is used.

Clearly, the development of a method for the generation of arrays of functional proteins, and in certain embodiments high density arrays of functional proteins, allows for the subsequent development of assay techniques to yield valuable information when applied to the system-wide study of protein function. These methods include, but are not limited to, screening for protein-protein interactions, screening for protein-polynucleotide interactions, screening for the substrates of protein kinases, screening for the protein targets of small molecules, and screening for molecules that disrupt or compete with a protein-biomolecule interaction. Although these methods of using protein microarrays are very powerful, the protein microarrays provided by the present invention may be used in any conventional assay in which proteins are screened. Other particularly useful methods include screening antibodies, or screening proteins with antibodies. Part of the power of the protein microarray is that it can be used to screen functional proteins using conventional methods with readily available reagents and standard equipment on a very large scale.

(1) Screening for Protein-Protein Interactions.

Currently, protein-protein interactions have only been investigated systematically on a genome-wide scale using the yeast two-hybrid system (Bartel et al. Nat. Genet. 12:72-77, 1996; Uetz et al Nature 403:623-627, 2000; each of which is incorporated herein by reference). While easy to implement and of great utility, this in vivo method suffers from several limitations. Proteins that function as transcriptional activators yield false positives when expressed as DNA binding domain fusions. False negatives are also encountered when proteins are displayed inappropriately or when the DNA binding domain fusions are produced in excess. Proteins that do not fold correctly in yeast are inaccessible by this technique and post-translational modifications (such as phosphorylation or glycosylation) cannot be controlled. Finally, it is impossible to control the environment during the experiment (e.g., ion concentration, presence or absence of cofactors, temperature, etc.). For these reasons, the high throughput identification of protein-protein interactions would benefit greatly from the development of in vitro methods of analysis.

In screening for protein-protein interactions or for protein targets of known proteins, any proteins may be used on the microarrays or in the assay method. Examples of proteins that may be used in the present invention include, but are not limited to, enzymes (e.g., proteases, kinases, synthases, synthetases), extracellular matrix proteins (e.g., keratin, elastin, proteoglycans), receptors (e.g., LDL receptor, amino acid receptors, neurotransmitter receptors, hormone receptors, adhesion molecules), signaling proteins (e.g., cytokines, insulin, growth factors), transcription factors (e.g., homeodomain proteins, zinc-finger proteins), and members of the immunoglobulin family (e.g., antibodies, IgG, IgM, IgE). As discussed above, in certain embodiments of special interest, the proteins printed on the microarrays are stable enough to be microarrayed and assayed. In certain embodiments, the proteins are derived from a recombinant source and are in a substantially pure form. In other embodiments, the proteins may be derived from cell lysates or may be collections of proteins. Direct protein-protein interactions (e.g., antibody-antigen) may be assayed for using this method, or secondary interactions through a third protein, a nucleic acid, a biological macromolecule, or a small molecule may be studied (e.g., FRB domain of FRAP and FKBP12). These protein-protein interactions may be detected via any method known in the art including fluorescence, radioactivity, immunoassay, etc. (for more detail on these methods, please see Ausubel et al., eds., Current Protocols in Molecular Biology, 1987; Sambrook et al. Molecular Cloning: A Laboratory Manual, 2nd Ed., 1989; each of which is incorporated herein by reference). In certain embodiment of special interest, the protein being used to probe the array is attached to a fluorescent compound, labeled with a radioactive isotope, or attached to a known epitope of an antibody. Certain exemplary embodiments will be described in more detail below.

In one embodiment, three pairs of proteins that are known to interact were selected: protein G and IgG (Bjorck et al. J. Immunol. 133:969-974, 1984; incorporated herein by reference); p50 (of the NFκB complex) and IΛBα. (Baeuerle et al. Science 242:540-546, 1988; incorporated herein by reference); and the FRB domain of FRAP and FKBP12 (Brown et al. Nature 369:756-758, 1994; Choi et al Science 273:239-242, 1996; each of which is incorporated herein by reference). While the first two interactions occur without any special requirements, the third interaction is dependent on the presence of the small molecule rapamycin (Brown et al. Nature 369:756-758, 1994; incorporated herein by reference). The first protein of each pair was arrayed in quadruplicate on a series of five identical aldehyde slides, with a distance of 250 μm between the centers of adjacent spots. After 3 hours, the slides were quenched and probed with different fluorescently labeled proteins.

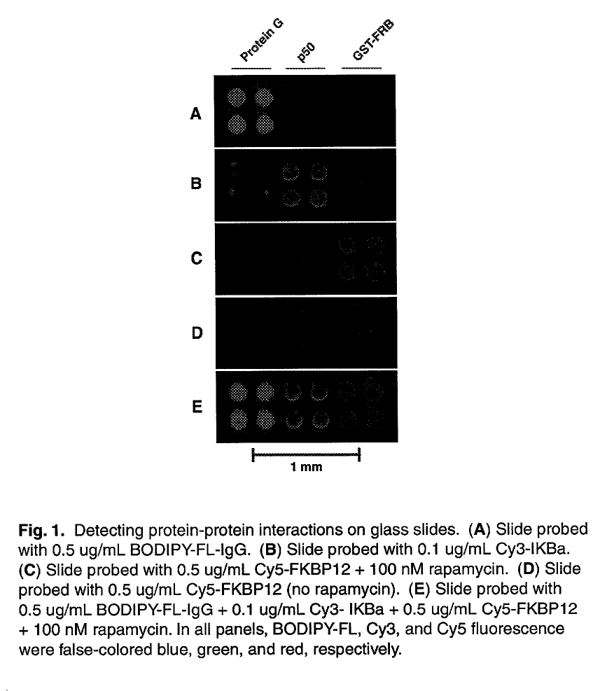

The slide in FIG. 1A was probed with BODIPY-FL-conjugated IgG, washed, and scanned with an ArrayWoRx fluorescence slide scanner (Applied Precision, Issaquah, Wash.) (BODIPY-FL fluorescence was false-colored blue). As anticipated, only the spots containing protein G were visible, indicating that the immobilized protein is able to retain its functional properties on the glass surface (at least with respect to its ability to interact specifically with proteins in solution). Similarly, the slide in FIG. 1B was probed with Cy3-conjugated IκBα. As anticipated, the Cy3 fluorescence (false colored green) localized to the p50-containing spots. To test the ability to modulate interactions by controlling the conditions in the buffer, two additional slides were probed with Cy5-conjugated FKBP12, either in the presence (FIG. 1C) or absence (FIG. 1D) of 100 nM rapamycin. As expected, the Cy5 fluorescence (false-colored red) was seen only when rapamycin was added. Since the three fluorophores used for these studies have non-overlapping excitation and emission spectra, these three interactions could be detected simultaneously (FIG. 1E).

To define the concentration range under which this assay can be performed, the concentration of both FRB (the immobilized protein) and Cy5-FKBP12 (the solution-phase protein; data not shown) were varied. At high concentrations of FRB (about 1 mg/ml), the fluorescence of the spots begins to saturate. Below this, the fluorescence scales linearly with decreasing concentrations of FRB. FIGS. 1, 2, 3, 5, and 7 employ a concentration of 100 μg/ml for the protein being spotted. This concentration is readily achievable in the context of high throughput protein expression/purification and can even be achieved using recently reported in vitro transcription/translation systems (Madin et al. Proc. Natl. Acad. Sci. USA 97:559-564, 2000; incorporated herein by reference).

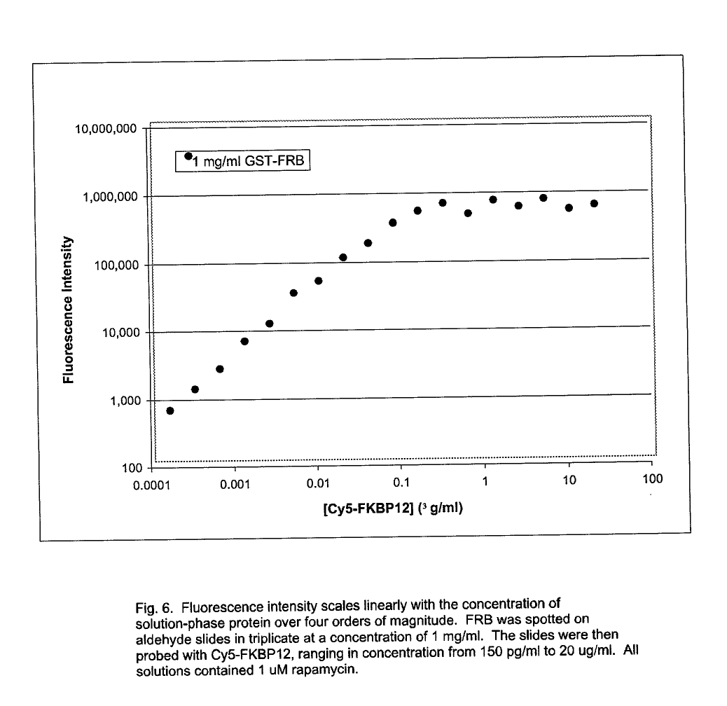

Much lower concentrations are needed for the solution-phase protein. In the case of Cy5-FKBP12, fluorescence of the resulting spots scales linearly with protein concentration over four orders of magnitude (see FIG. 6). Specific binding can easily be detected using as little as 150 pg/ml Cy5-FKBP12 (˜12.5 pM). This, of course, is easily accessible using purified protein, but is also accessible using fluorescently labeled proteins from cell lysates. This means that specific protein-protein interactions, once defined, may potentially be exploited to quantify protein abundance and modification in whole cells or tissues (“profiling” at the protein level).

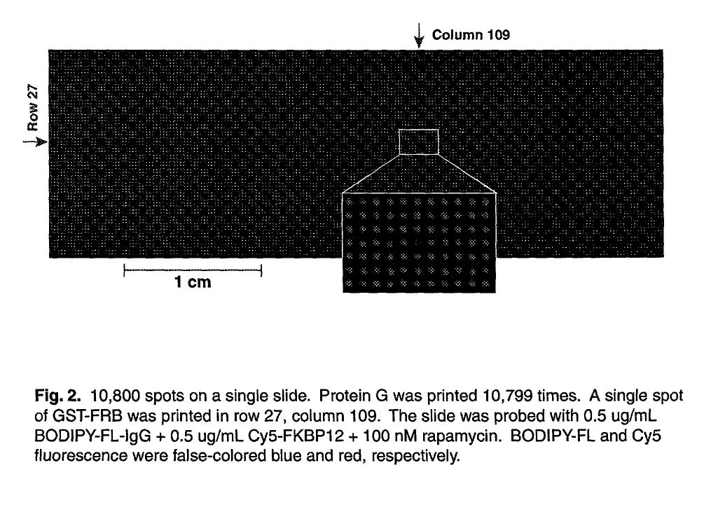

At the spot density employed for these studies, it is possible to fit over 10,000 samples in about half the area of a standard 2.5 cm×7.5 cm slide. To investigate the feasibility of detecting a single specific interaction in this larger context, a slide was prepared containing 60 rows and 180 columns of spatially separated spots resulting in 10,800 total spots. Protein G was spotted 10,799 times on this slide, with a single spot of GST-FRB in row 27, column 109. The slide was then probed with a mixture of BODIPY-FL-IgG and Cy5-FKBP12, with 100 nM rapamycin included in the buffer. FIG. 2 shows the single FRB spot, clearly visible in the sea of protein G spots.

(2) Screening for the Substrates of Protein Kinases.

While it is of great value to identify stable protein-protein interactions in a system such as a cell or tissue, it is equally important to define the transient interactions that occur between enzymes and their substrates, particularly peptide and/or protein substrates. Enzymes that have proteins as substrates include, but are not limited to, phosphatases, kinases, acetylases, deacetylases, methylases, demethylases, proteases, and other post-translation modification enzymes (e.g., prolyl hydroxylase, lysyl hydroxylase).

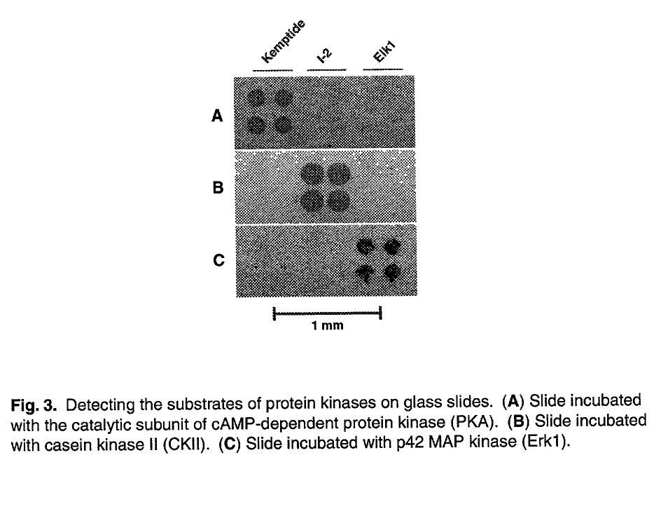

To give but one example, most signal transduction networks are composed of numerous kinases and the proteins that they phosphorylate. Protein microarrays offer an ideal system for the rapid and parallel identification of the substrates of protein kinases. A microarray of proteins can be incubated with a purified kinase in the presence of ATP, and the substrates identified by detecting their phosphorylation. In but one exemplary system, three different kinase/substrate pairs were chosen: cAMP-dependent protein kinase (PKA) and “Kemptide” (a peptide substrate for PKA) (Kemp et al. J. Biol. Chem. 252:4888-4894, 1977; incorporated herein by reference); casein kinase II (CKII) and protein phosphatase inhibitor 2 (I-2) (DePaoli-Roach J. Biol. Chem. 259:12144-12152, 1984; incorporated herein by reference); and p42 MAP kinase (Erk2) and Elk1 (Marais et al. Cell 73:381-393, 1993; incorporated herein by reference). The protein substrates of each pair were spotted in quadruplicate onto a series of three identical BSA-NHS slides with a distance of 250 μm between the centers of adjacent spots. Following a three-hour incubation, each slide was quenched with glycine and incubated with a different kinase in the presence of ³³P-γ-ATP (FIG. 3).

While isotopic labeling of the protein spots is the most direct way to identify phosphorylation, the challenge lies in detecting the radioactive decay. Neither X-ray film nor conventional phosphorimagers offer sufficient spatial resolution to visualize the 150-200 μm diameter spots. Utilizing the technique of isotopic in situ hybridization (Wilcox et al. Methods Enzymol. 124:510-533, 1986; incorporated herein by reference), the slides were dipped in a photographic emulsion and developed manually, resulting in the deposition of silver grains directly on the glass surface. The slides were then visualized using an automated light microscope (DeltaVision microscope, Applied Precision, Issaquah, Wash.), and the individual frames were stitched together to yield the final images shown in FIG. 3.

The slide in FIG. 3A was incubated with PKA. As expected, only the substrate for PKA (Kemptide) was phosphorylated. Our ability to detect the specific phosphorylation of a short peptide indicates that peptide libraries, prepared by parallel synthesis, may be screened at very high spatial densities using this technique. Since isotopic in situ hybridization is known to be quantitative, the best substrates in a library of peptides can be readily identified. It should be noted that peptides can also be assembled directly on glass surfaces by parallel synthesis using photolithographic masks (Fodor et al. Science 251:767-773, 1991; incorporated herein by reference) or on membranes using robotically controlled spot synthesis (Frank Tetrahedron 48:92179232, 1992; incorporated herein by reference). Moreover, spot synthesis has been used successfully to define the substrate specificity of several protein kinases (Toomik et al. Pept. Res. 9:6-11, 1996; incorporated herein by reference). An attractive feature of contact printing, however, is the fact that full-length proteins can be immobilized as well as peptides. FIGS. 3B and 3C show slides incubated with CKII and Erk2, respectively. As with PKA, only the specific substrates of these kinases (I-2 and Elk1, respectively) were phosphorylated.

(3) Screening for the Targets of Small Molecules.

In yet another application of the usefulness of protein microarrays, protein microarrays were used to identify protein-small molecule interactions. Small molecules are particularly useful given the fact that most pharmaceutical agents are small molecules. Small molecules have been identified that alter the function of the proteins to which they bind. For example, colchicine has been found to inactivate the function of tubulin, and steroid hormones have been found to activate the transcriptional properties of nuclear hormone receptors. With the advent of combinatorial chemistry and the expansion of natural product collections, more and more compounds are routinely screened for biological activity. In many cases, cell-based screens are used and active compounds identified by their ability to induce a desired phenotype. Once a “hit” is obtained, the daunting task of target identification remains. To what protein does the active small molecule bind? Does the compound bind with low affinity to any other proteins and if so, can this information be exploited to eliminate negative side effects as the compound is developed into a drug? Unfortunately, current methods of target identification are slow and laborious. Affinity-based purification and microsequencing of the protein target is often the method of choice. While techniques employing phage display (Caterina et al. Nature 389:816-824, 1997; Sche et al. Chem. Biol. 6:707-716, 1999; each of which is incorporated herein by reference), the yeast three-hybrid system (Licitra et al. Proc. Natl. Acad. Sci. USA 93:12817-12821, 1996; incorporated herein by reference), membrane-based plaque lifts (Tanaka et al. Mol. Pharmacol 55:356-363, 1999; incorporated herein by reference), and in vivo expression cloning (Caterina et al. Nature 389:816-824, 1997; incorporated herein by reference) have been described, they all suffer from the common limitations imposed by employing random cDNA libraries. As an alternative to these techniques, microarray-based assays were developed that employ purified, full-length, and correctly folded proteins.

In the method of screening for targets of small molecules, any protein may be printed on a microarray. The proteins are substantially pure and are derived from a recombinant source. In other embodiments, the proteins at each spot of the array are collections of proteins, preferably from 2-10 proteins. It will be appreciated that any small molecule may be used in the assay. In certain embodiments, the small molecule itself can be assayed for, or the small molecule can be tagged to allow for easy detection of the small molecule. In certain embodiments, the small molecule is covalently linked to a fluorescent tag (e.g., Alexa₄₈₈, Cy5, Cy3, BODIPY-FL). In yet another embodiment, the small molecule is labeled with a radioactive isotope for easy detection.

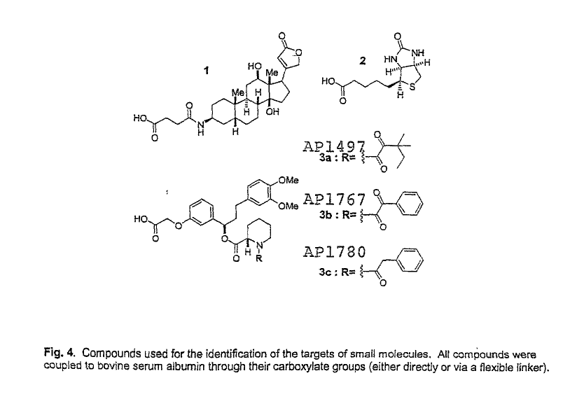

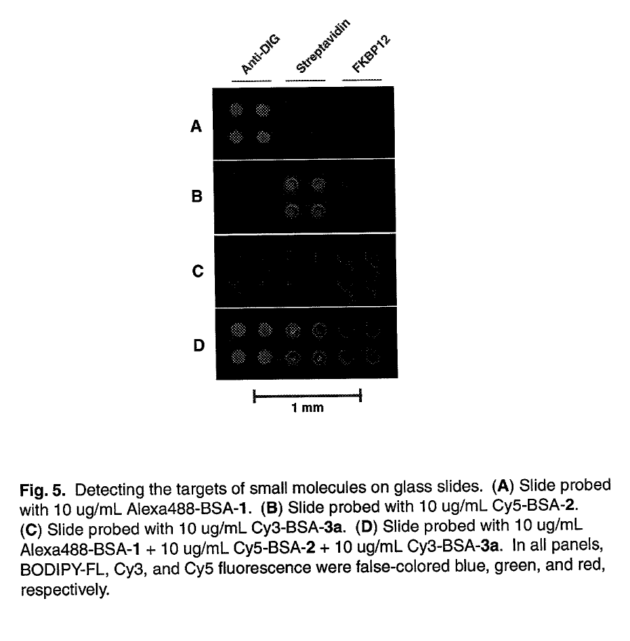

To give but a few non-limiting examples, three unrelated molecules for which specific protein receptors are available were chosen. Compound 1 (FIG. 4) is a derivative of the steroid digoxigenin and is recognized by the mouse monoclonal antibody anti-DIG (Mouse anti-digoxigenin IgG clone 1.71.256 (Boehringer Mannheim, Indianapolis, Ind.)). Compound 2 is the vitamin biotin, which is recognized by the bacterial protein streptavidin (Chaiet et al. Arch. Biochem. Biophys. 106:1-5, 1964; incorporated herein by reference). Finally, compound 3a is a synthetic pipecolyl α-ketoamide, which was designed to be recognized by the human immunophilin FKBP12 (Holt et al. J. Am. Chem. Soc. 115:9925-9938, 1993; incorporated herein by reference). All three proteins were spotted in quadruplicate on four aldehyde slides, using the same spot density employed in previous experiments. After blocking the slides with BSA, each slide was probed with a different small molecule. Rather than labeling the compounds directly, each molecule was coupled to BSA that had previously been labeled with a fluorophore. Thus, the slide in FIG. 5A was probed with an Alexa₄₈₈-BSA-1 conjugate and scanned for Alexa₄₈₈ fluorescence (false-colored blue). As anticipated, only the anti-DIG spots were fluorescent. Similarly, the slides in FIGS. 5B and 5C were probed with Cy5-BSA-2 and Cy3-BSA-3a, respectively. In each case, the fluorescence localized to the appropriate spots. Since the three fluorophores used for these studies have non-overlapping excitation and emission spectra, all three interactions could be detected simultaneously (FIG. 5D).

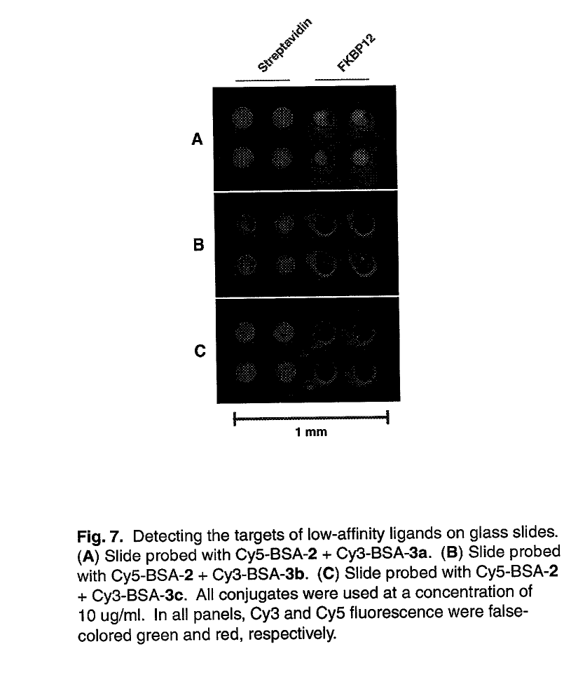

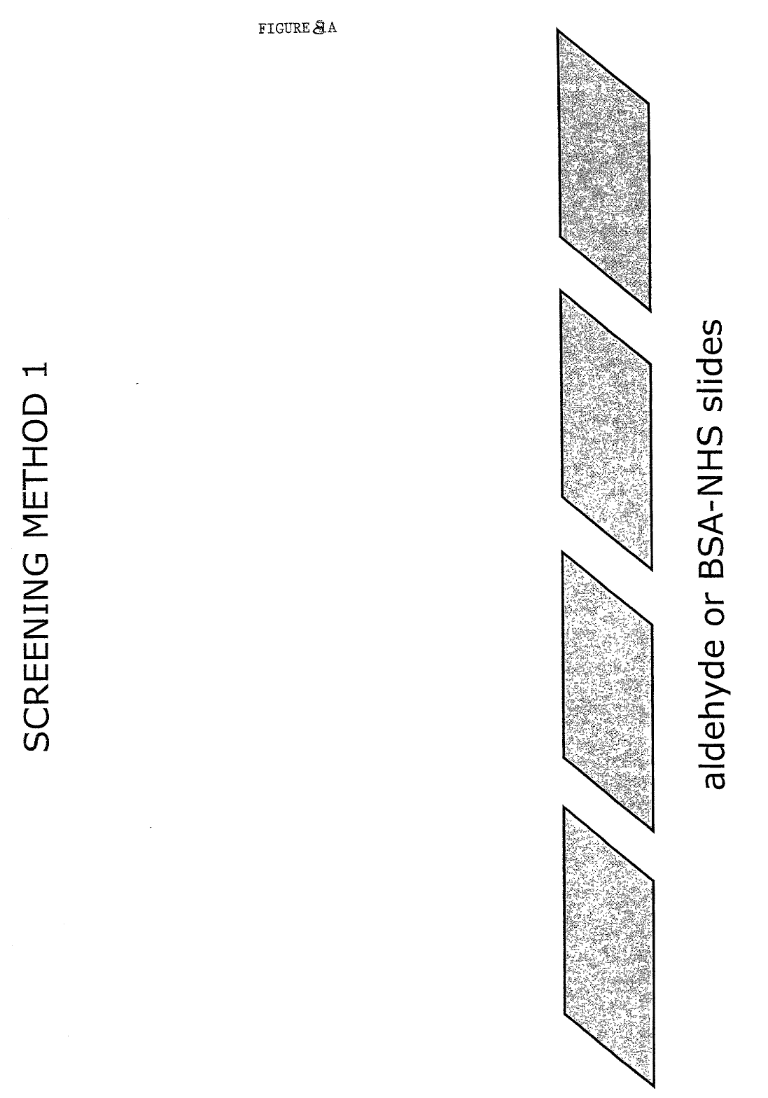

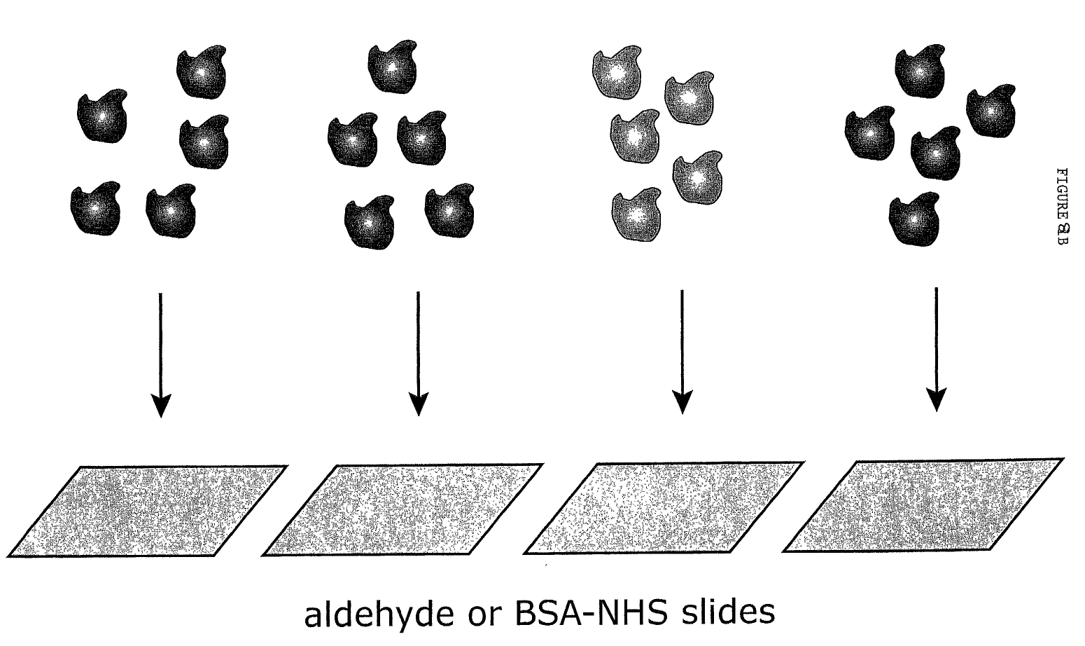

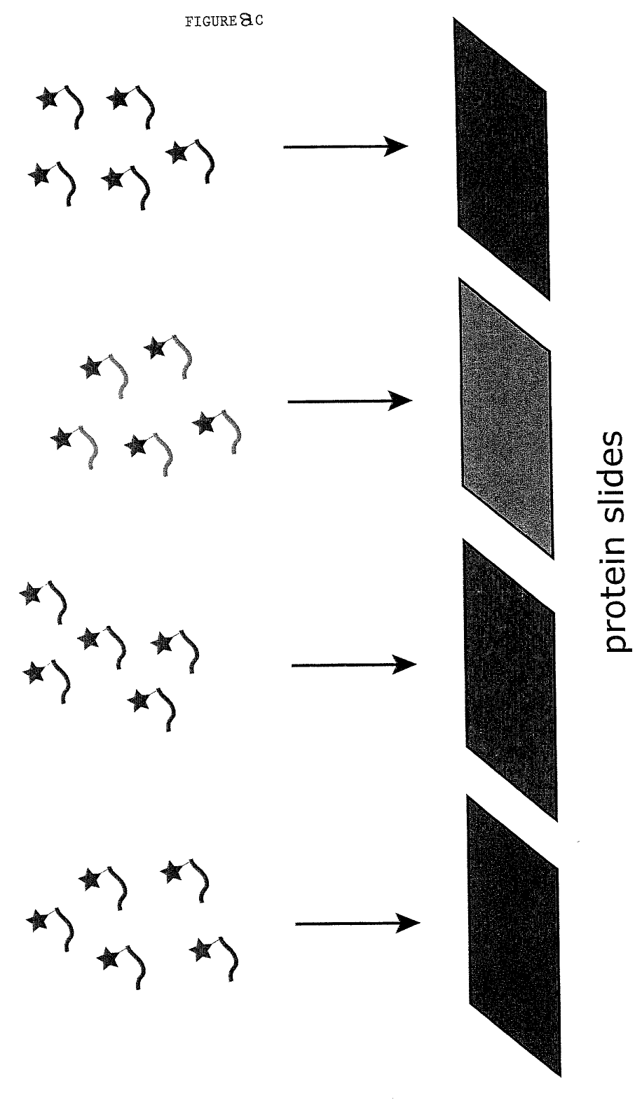

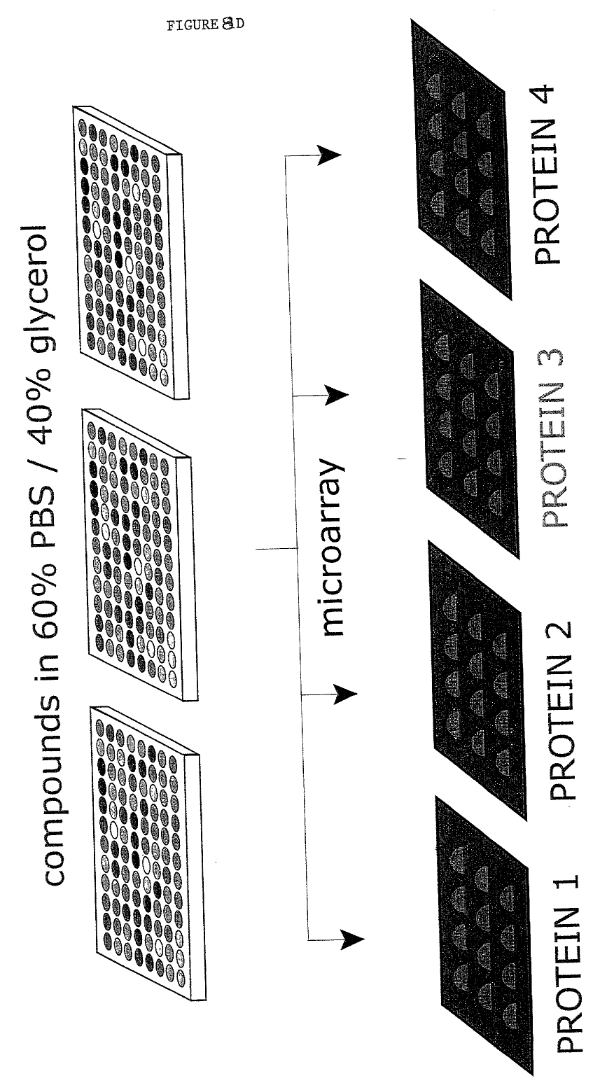

To investigate the ability to detect low-affinity interactions, Cy3-BSA conjugates of compounds 3a, 3b, and 3c (FIG. 4) (dissociation constants for FKBP 12 of 8.8 nM, 140 nM, and 2.6 μM, respectively) were prepared. When three identical slides displaying FKBP12 were probed in parallel, spots with comparable fluorescent intensities were obtained for all three conjugates (see FIG. 7 and the Examples below). This means that interactions in the micromolar range can easily be observed. The fact that the intensity of the fluorescence did not vary significantly as the affinity of the interaction was lowered can be attributed to the multivalency of the BSA conjugates (avidity effects). It has been previously shown that when compounds 3a, 3b, and 3c are immobilized on a glass surface and subsequently probed with Cy5-labeled FKBP 12 (a monomeric protein), the intensity of the fluorescence correlates very well with the affinity of the interaction (MacBeath et al. J. Am. Chem. Soc. 121:7967-7968, 1999; incorporated herein by reference). Thus, by controlling the valency of the probe, one can choose whether to observe differences in affinity or to favor the detection of low-affinity interactions. The combination of these two approaches may prove useful in the identification of both primary and secondary drug targets.

(4) Screening for Competitors of Ligand Binding.

In another embodiment, protein microarrays may be used to identify molecules that can displace a ligand from its receptor. Targets immoblized on a solid support in a uniform layer are incubated with a known ligand of the target (which may or may not be labeled). The target/ligand pair on the microarray is then incubated with microarrayed molecules to allow the molecule to compete with the ligand for binding to the immobilized target.

The target may be any appropriate biomolecule including proteins, glycoproteins, polynucleotides, carbohydrates, lipids, etc. The target may also be a mixture of proteins or even a whole cell. The target may be a natural cell or a cell engineered to overexpress a protein, including but not limited to a cell surface receptor. In certain embodiments, the target is a protein. The target is attached to a solid support through a covalent or non-covalent interaction. Preferably, the interaction is a covalent one. The target is attached to the solid support using methods described herein. Following coating of the surface of the solid support, the support is washed and then incubated with a known ligand of the target. The ligand may be a small molecule, peptide, protein, biomolecule, polynucleotide, polymer, lipid, carbohydrate, etc. In certain embodiments, the ligand is a peptide or small molecule, which ligand may in certain embodiment be labeled. The ligand or ligands may be printed as an array onto the solid support. Examples of labels include, but are not limited to, a fluorophore, a radioactive isotope, hapten, affinity reagent, chromophore, and oligonucleotide. In alternative embodiments of special interest, the label is a fluorophore (e.g., cyanine 5). After allowing the ligand to bind to its target, the support is washed to remove any unbound ligand.

It will be appreciated that a variety of molecules can be screened for their ability to disrupt a ligand/target interaction or compete with a ligand for binding to the target attached to the solid support. These molecules include, but are not limited to, small molecules, biomolecules, proteins, peptides, glycoproteins, polynucleotides, carbohydrates, lipids, etc. The molecules are prepared by dissolving them in an appropriate buffer that includes a humectant that prevents evaporation of small droplets. In certain embodiments, the buffer used is phosphate-buffered saline with 40% glycerol added as a humectant. In certain embodiments, the molecules are stored in microtiter plates, such as 384-well plates. Using a robot, the molecules are arrayed onto the solid support at a high spatial density. In certain embodiments, the molecules are arrayed at a density at least 1000 spots per cm², and in other embodiments are at least 1500 spots per cm². Each droplet of the molecule solution has a very small volume (e.g., 10 nL, 5 nL, 1 nL, 0.5 nL). The spots are spatially separated so that each droplet forms a separate “reaction vessel” and there is little to no contamination between spots. The microarray is then allowed to incubate for an appropriate amount of time and at an appropriate temperature, as would be appreciated by one of skill in this art, to enable the molecules being screened to compete with the ligand for binding to the immobilized target. After the appropriate incubation period, the solid support is washed and subsequently analyzed. The analysis detects whether or not any of the molecules being screened are able to displace the bound ligand. Any appropriate detection method may be used. Labeled ligands can be detected using, for example, fluorescence, luminescence, radioactivity, or other appropriate method. Non-labeled ligands may be detected with other technologies, including mass spectometry, surface plasmon resonance, conductivity, or other appropriate methods. In certain embodiments, fluorescence detection of a ligand labeled with a fluorophore is used in the inventive method. A decreased level of the ligand in any given spot indicate that the solution-phase molecule being screened was able to compete with the ligand for binding to the immobilized target.

The foregoing methods are just a few of the many screening methods for which high-density microarrays of functional proteins can be used. Others of skill in this art will certainly appreciate other methods for which the inventive arrays can be utilized.

While traditional biochemical methods have yielded invaluable insight into protein function on a case-by-case basis, they cannot realistically be applied to the study of every protein in a cell, tissue, or organism. If the functions of proteins are to be studied on a larger level, miniaturized assays that can be performed in a highly parallel format must be utilized. Although it is certainly a daunting task to express and purify thousands of different proteins, the effort is made worthwhile if these proteins can be assayed both simultaneously and repeatedly. By fabricating protein microarrays, we can fulfill both these criteria, facilitating the in vitro study of protein function on a genome-wide level.

This and other aspects of the present invention will be further appreciated upon consideration of the following Examples, which are intended to illustrate certain particular embodiments of the invention but are not intended to limit its scope, as defined by the claims.

### EXAMPLES

Materials and Methods

Chemically Derivatized Glass Slides.

SMA slides, displaying aldehydes on their surface, were purchased from TeleChem International (Cupertino, CA). BSA-NHS slides, displaying activated amino and carboxyl groups on the surface of an immobilized layer of bovine serum albumin (BSA), were fabricated as follows. 10.24 g NN′-disuccinimidyl carbonate (100 mM) and 6.96 ml N,N-diisopropylethylamine (100 mM) were dissolved in 400 ml anhydrous N,N-dimethylformamide (DMF). 30 CMT-GAP slides (Coming Incorporated, Corning, N.Y.), displaying amino groups on their surface, were immersed in this solution for 3 hr at room temperature. The slides were rinsed twice with 95% ethanol and then immersed in 400 ml of phosphate buffered saline (PBS), pH 7.5 containing 1% BSA (w/v) for 12 hr at room temperature. The slides were rinsed twice with ddH₂O, twice with 95% ethanol, and centrifuged at 200 g for 1 min to remove excess solvent. The slides were then immersed in 400 ml DMF containing 100 mM N,N′-disuccinimidyl carbonate and 100 mM N,N-diisopropylethylamine for 3 hr at room temperature. The slides were rinsed four times with 95% ethanol and centrifuged as above to yield BSA-NHS slides. The slides were stored in a desiccator under vacuum at room temperature for up to two months without noticeable loss of activity.

Arraying Proteins on Glass Slides.

Proteins were dissolved in 40% glycerol, 60% PBS, pH 7.5, at a concentration of 100 μg/ml unless otherwise indicated. For FIGS. 1, 5, 6, and 7, the proteins were spotted on SMA slides using a GMS 417 Arrayer (Affymetrix, Santa Clara, Calif.). Following a 3 hr incubation in a humid chamber at room temperature, the slides were inverted and dropped onto a solution of PBS, pH 7.5 containing 1% BSA (w/v). After 1 min, the slides were turned right side up and immersed in the BSA solution for 1 hr at room temperature with gentle agitation. Following a brief rinse in PBS, the slides were ready for further processing (see below).

For FIG. 3, the proteins were spotted on BSA-NHS slides using a GMS 417 Arrayer. Following a 3 hr incubation in a humid chamber at room temperature, the slides were inverted and dropped onto a solution of PBS, pH 8.0, containing 500 mM glycine. After 1 min, the slides were turned right side up and immersed in the glycine solution for 1 hr at room temperature with gentle agitation. The slides were then ready for further processing (see below).

For FIG. 2, the proteins were spotted on a single SMA slide using a split pin arrayer constructed following directions on P. Brown's web page (http://cmgm.stanford.edu/pbrown/; incorporated herein by reference). Following a 3 hr incubation at room temperature, the slide was processed using the procedure employed for the SMA slides described above.

Screening for Protein-Protein Interactions.

Protein G was from Pierce (Rockford, Ill.), and BODIPY-FL-Goat-anti-Mouse IgG was from Molecular Probes (Eugene, Oreg.). IκBα and p50 were kindly provided by T. Maniatis (Harvard University, Cambridge, Mass.), and GST-FRB and (His)₆-FKBP12 were produced recombinantly in Escherichia coli. IκBα and (His)₆-FKBP12 were labeled with Cy3 and Cy5, respectively, using monofunctional reactive dye from Amersham Pharmacia Biotech (Newark, N.J.) and following the recommended protocol.

For FIG. 1, protein G, p50, and GST-FRB were spotted in quadruplicate on SMA slides and processed as described above. To probe the slides, the labeled proteins were diluted into PBS, pH 7.5, supplemented with 0.1% Tween-20 (v/v) and 1% BSA (w/v). BODIPY-FL-IgG was used at a concentration of 0.5 μg/ml, Cy3-IκBα was used at a concentration of 0.1 μg/ml, and Cy5-FKBP12 was used at a concentration of 0.5 μg/ml. 0.55 ml of protein solution was applied to the slide using a PC500 CoverWell incubation chamber from Grace Biolabs (Bend, Oreg.). Following a 1 hr incubation at room temperature, the slides were rinsed with PBS and then washed 3 times for 3 min each with PBST (PBS supplemented with 0.1% Tween-20). The slides were rinsed twice with PBS and centrifuged at 200 g for 1 min to remove excess buffer.

To visualize fluorescence, the slides were scanned using an ArrayWoRx fluorescence slide scanner (Applied Precision, Issaquah, Wash.). The scanner works by imaging successive 2.5×2.5 mm sections of a slide using excitation and emission filters coupled with a magnifying lens and CCD camera. The resulting panels are then stitched together to form one large image. The slides were visualized at 5 μm resolution, using CCD camera integration times ranging from 1 to 5 sec depending on the fluorophore. The emitted light was false-colored blue, green, and red to correspond to BODIPY-FL, Cy3, and Cy5, respectively. For all images, the intensity of the color was scaled linearly, with black corresponding to the background fluorescence of the slide and pure color corresponding to the brightest pixels in the image.

For FIG. 2, protein G and FRB were spotted on an aldehyde slide, probed with BODIPY-FL-IgG +Cy5-FKBP12+100 nM rapamycin, and visualized with an ArrayWoRx fluorescence slide scanner, all as described above. For FIG. 6, FRB (1 mg/ml) was spotted in triplicate on twelve separate areas of two aldehyde slides. The areas were then separated by drawing lines between them with a hydrophobic pen (PAP PEN from Newcomer Supply, Middleton, Wis.). The slides were processed as described above. To probe the slides, Cy5-FKBP12 was serially diluted 2-fold into PBST containing 1% BSA (w/v) and 1 μM rapamycin. 30 μl of each dilution were applied to separate sections of the slides. Following a one hour incubation at room temperature, the slides were washed as described above and scanned with a GenePix 4000A microarray scanner (Axon Instruments, Foster City, Calif.). The fluorescence intensity of each spot was taken as the median intensity of the spot minus the median intensity of the local background. In order to span the full range of intensities observed at different concentrations of Cy5-FKBP12, the two slides were scanned at different sensitivity settings (PMT voltage), and the data were scaled to adjust for this difference.

Screening for Substrates of Protein Kinases.

All proteins used for these studies were purchased from New England Biolabs (Beverly, Mass.). EasyTides γ-³³P-adenosine 5′-triphosphate (γ-³³P-ATP) was from NEN Life Science Products (Boston, Mass.). NTB-2 autoradiography emulsion, Dektol developer, and Fixer were from Eastman Kodak Company (Rochester, N.Y.).

Kemptide, I-2, and Elk1 were spotted in quadruplicate on BSA-NHS slides and processed as described above. The slides were then washed 3 times for 10 min each with Wash Buffer (WB; 20 mM Tris, 150 mM NaCl, 10 mM EDTA, 1 mM EGTA, 0.1% Triton X-100, pH 7.5). The slides were subsequently washed once for 10 min with Kinase Buffer (KB; 50 mM Tris, 10 mM MgCl₂, 1 mM DTT, pH 7.5), incubated for 10 min with KB supplemented with 100 μM ATP, and washed for an additional 10 min with KB. The slides were then incubated for 1 hr at room temperature with 200 μl of kinase solution, applied to the slides under a PC200 CoverWell incubation chamber (Grace Biolabs). The kinase solution was composed of the recommended buffer for each kinase supplemented with the recommended amount of ATP, 2 μl of y-³³P-ATP (20 μCi), and 2 μl of purified enzyme (10 units of cAMP-dependent protein kinase (catalytic subunit), 1000 units of casein kinase II, or 100 units of Erk2). Following the 1 hour incubation, the slides were washed 6 times for 5 min each with WB, twice for 5 min each with WB lacking Triton X-100, and 3 times for 3 min each with ddH₂O. The slides were then centrifuged at 200 g for 1 min to remove excess water.

To visualize the radioactive decay, NTB-2 autoradiography emulsion was melted at 45° C. for 45 min in a dark room. The slides were dipped in the emulsion for 3 sec and allowed to dry vertically at room temperature for 4 hr. The slides were then sealed in a β-radiation box with desiccant and incubated in the dark at 4° C. for 4 to 10 days. The slides were subsequently developed by immersing them successively in Dektol developer for 2 min, ddH₂O for 10 sec, Fixer for 5 min, and ddH₂O for 5 min. To visualize the slides, successive images were taken using a DeltaVision automated microscope (Applied Precision) in DIC mode, and the individual panels were stitched together to form a single larger image. The same settings were used for all three slides.

Screening for Targets of Small Molecules.

Mouse anti-digoxigenin IgG clone 1.71.256 (Anti-DIG) was from Boehringer Mannheim (Indianapolis, Ind.), streptavidin was from Pierce, and bovine serum albumin (BSA) was from Sigma (St. Louis, Mo.). Alexa₄₈₈-BSA was prepared using an Alexa Fluor 488 protein labeling kit from Molecular Probes. Alexa₄₈₈-BSA-Dig was prepared by labeling Alexa₄₈₈-BSA with 3amino-3-deoxydigoxigenin hemisuccinamide, succinimidyl ester (Molecular Probes). Cy3-BSA and Cy5-BSA were prepared by labeling BSA with Cy3 and Cy5 monofunctional reactive dyes (Amersham Pharmacia Biotech). Cy5-BSA-biotin was prepared by labeling Cy5-BSA with Sulfo-NHS-LC-Biotin (Pierce). Cy3-BSA-maleimide was prepared by labeling Cy3-BSA with Sulfo-GMBS (Pierce). All labeling reactions were performed according to the recommended protocols.

Cy3-BSA-AP1497, Cy3-BSA-AP1767, and Cy3-BSA-AP1780 were prepared as follows. AP1497, AP1767, and AP1780 were kindly provided by D. Holt (Ariad Pharmaceuticals, Cambridge, Mass.). Each compound was coupled to polystyrene beads via a 6-carbon linker and 4-methoxytrityl-protected cysteine according to our previously published protocol (http://www-schreiber.chem.harvard.edu/home/protocols/SMP.html; incorporated herein by reference). For each compound, about 15 beads were incubated in 100 μl of a 17:2:1 mixture of chloroform, trifluoroacetic acid, and triethylsilane for 2 hr at room temperature. The cleavage solution was then removed in vacuo, yielding about 750 nmol of each compound. About 165 equivalents of thiol-labeled small molecule was incubated with Cy3-BSA-maleimide in PBS for 6 hours at room temperature. Following a 1 hour incubation with 200 mM 2-mercaptoethanol, the conjugates were dialyzed extensively against PBS, yielding Cy3-BSA-AP1497, Cy3-BSA-AP1767, and Cy3-BSAAP 1780.

Anti-DIG, streptavidin, and FKBP12 were spotted in quadruplicate on SMA slides and processed as described above. To probe the slides, the doubly labeled BSA conjugates were diluted into PBST supplemented with 1% BSA (w/v) at a concentration of 10 μg/ml. 0.55 ml of protein solution was applied to the slide, using a PC500 CoverWell incubation chamber. Following a 1 hr incubation at room temperature, the slides were rinsed with PBS and then washed 3 times for 3 min each with PBST. The slides were rinsed twice with PBS, centrifuged at 200 g for 1 min to remove excess buffer, and imaged on an ArrayWoRx fluorescence slide scanner as above.

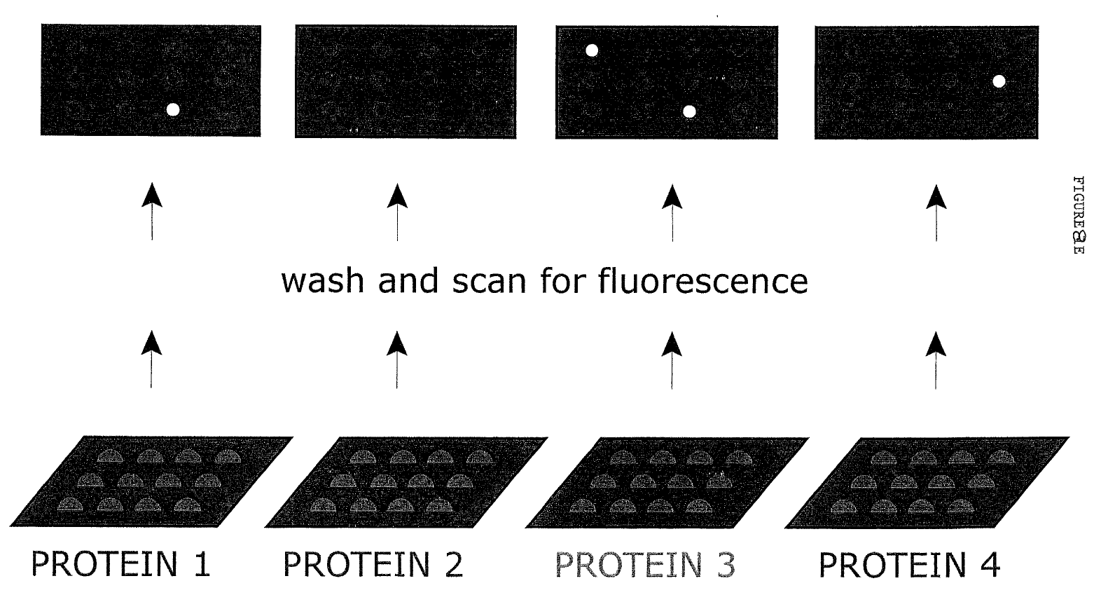

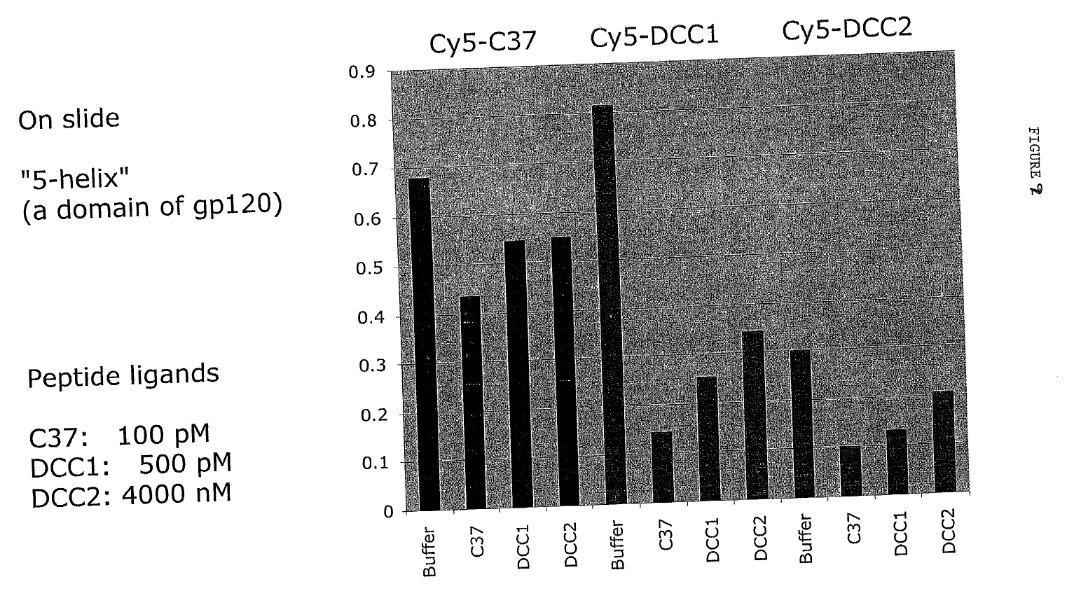

Screening for competitors of ligand binding (FIG. 8). SuperAldehyde slides were coated at room temperature with a solution of “5-helix” dissolved in phosphate buffered saline, pH 7.5 (PBS), at a concentration of 0.1 mg/ml. “5-helix” is a portion of the HIV protein gp41 and has been previously described by Root et al. in Science 291:884-888, 2001; which is incorporated herein by reference. After 1 hour, the slides were immersed in a solution of PBS/Tween-20 (0.1%) [PBST] +1% BSA, at room temperature for one hour to quench all the unreacted sites on the slide. After 1 hour, the slides were rinsed with PBST and then incubated for 1 hour at room temperature with either 10 nM of C37-H6 (GGHTTWMEWDREINNYTSLIHSLIEESQNQQEKNEQELLGGHHHHHH), 1 μM or 10 nM JN-DCC1 (GGHTTWMELDREINNYTSLIHSLIEESQNQQEKNEQELL), or 10 nM JN-DCC2 (GGHTTWMEADREINNYTSLIHSLIEESQNQQEKNEQELL) (Chan et al. Proc. Natl. Acad. Sci. USA 95:15613-15617, 1998; incorporated herein by reference). All three of these peptides are ligands for 5-helix. Of the three peptides, C37-H6 binds with the highest affinity, JN-DCC 1 with the second highest affinity, and JN-DCC2 with the lowest affinity. After the 1 hour incubation, the slides were washed with distilled water and centrifuged to remove excess buffer. To test the stability of these slides, they were then left at room temperature in a humid chamber for 24 hours before further processing After 24 hours, the slides were placed on a GMS417 Arrayer (Affymetrix) and unlabled peptides (C37-H6, JN-DCC1, and JN-DCC2), present in separate wells of a 96-well plate were arrayed onto the slides. The peptides were present at a concentrations of 10 μM, 1 μM, 0.1 μM, and 0.01 μM in 10% glycerol, 62% hydridization mix (1 M guanidine hydrochloride, 83.3% PBST, pH 7.5) with the remaining 28% being composed of peptide in PBST/1% BSA. A negative control (i.e., no peptide) was also included. The samples were arrayed at a spatial density of 250 μm center to center, which corresponds to a density of 1600 spots per cm². Each peptide, as well as the negative control, was spotted six times. After a 1 hour incubation, the slides were washed three times for 3 minutes each with PBS containing 0.1% Tween-20 and also including 1 M guanidine hydrochloride, rinsed once with distilled water, and centrifuged to remove excess buffer. The slides were then scanned with a GenePix 4000A slide scanner (Axon Instruments), and the fluorescence in each spot was quantified with the GenePix software that accompanies the scanner. FIG. 9 shows the average intensity of the spots for each peptide.

### Other Embodiments

Those of ordinary skill in the art will readily appreciate that the foregoing represents merely certain preferred embodiments of the invention. Various changes and modifications to the procedure and compositions described above can be made without departing from the spirit or scope of the present invention, as set forth in the following claims.

What is claimed is:

1. An array comprising spots of at least one type of protein attached to a solid support, wherein the density of said array of proteins is at least 1000 spots per cm².

2. The array of claim 1, wherein the proteins are functional.

3. The array of claim 1, wherein a substantial fraction of the proteins are functional.

3. The array of claim 1, wherein the array comprises proteins that are properly folded into their natural conformation.

4. The array of claim 1, wherein the density of the array is at least 1500 spots per cm².

5. The array of claim 1, wherein the proteins are attached to the solid support through a non-covalent interaction.

6. The array of claim 1, wherein the proteins are attached to the solid support through a covalent interaction.

7. The array of claim 5 or 6, wherein said interaction is characterized in that the linkage is robust enough so that the compounds are (1) not inadvertently cleaved during subsequent manipulation steps and (2) inert so that the functionalities employed do not interfere with subsequent manipulation steps.

8. The array of claim 5 or 6, whereby the interaction results in a substantial fraction of the arrayed proteins being functional.

9. The array of claim 6, wherein the covalent interaction is a Schiff's base linkage.

10. The array of claim 6, wherein the covalent interaction is generated by a transacylation reaction between the protein and a chemical moiety on the solid support.

11. The array of claim 6, wherein the covalent interaction is generated by a Michael addition reaction.

12. The array of claim 6, wherein the covalent interaction is a disulfide bond.

13. The array of claim 5, wherein the non-covalent interaction is an epitope-antibody interaction.

14. The array of claim 5, wherein the non-covalent interaction is a poly-histidine-metal cation interaction.

15. The array of claim 5, wherein the non-covalent interaction is nonspecific.

16. The array of claim 5 or 6, wherein the interaction between the protein and the solid support occurs through an interaction with a non-natural polymeric matrix.

17. The array of claim 16, wherein the polymeric matrix is dextran.

18. The array of claim 16, wherein the matrix is less than 500 nm thick.

19. The array of claim 1, wherein the space between the spots of the array is occupied by a blocking agent.

20. The array of claim 19, wherein the blocking agent is a protein.

21. The array of claim 19, wherein the blocking agent is selected from the group consisting of bovine serum albumin, caseine, and nonfat milk.

22. The array of claim 19, wherein the blocking agent is a small molecule.

23. The array of claim 19, wherein the blocking agent is selected from the group consisting of glycine, ethanolamine, and ethylenediamine.

24. The array of claim 1, wherein the solid support is glass.

25. The array of claim 1, wherein the solid support is a polymer.

26. The array of claim 1, wherein the solid support comprises a metal surface.

27. The array of claim 1, wherein the solid support comprises a self-assembled monolayer.

28. An array comprising spots of at least one type of protein attached to bovine serum albumin (BSA) coated onto a solid support, wherein the density of said array of proteins is at least 1000 spots per cm².

29. The array of claim 28, wherein the proteins are functional.

30. The array of claim 28, wherein a substantial fraction of the proteins are functional.

31. The array of claim 28, wherein the array comprises proteins that are properly folded into their natural conformation.

32. The array of claim 28, wherein the density of the array is at least 1500 spots per cm².

33. The array of claim 28, wherein the proteins are attached to the BSA through a non-covalent interaction.

34. The array of claim 28, wherein the proteins are attached to the BSA through a covalent interaction.

35. The array of claim 33 or 34, wherein said interaction is characterized in that the linkage is robust enough so that the compounds are (1) not inadvertently cleaved during subsequent manipulation steps and (2) inert so that the functionalities employed do not interfere with subsequent manipulation steps.

36. The array of claim 33 or 34, whereby the interaction results in a substantial fraction of the arrayed proteins being functional.

37. The array of claim 34, wherein the covalent interaction is a Schiff's base linkage.

38. The array of claim 34, wherein the covalent interaction is generated by a transacylation reaction between the protein and a chemical moiety on the BSA.

39. The array of claim 34, wherein the covalent interaction is generated by a Michael addition.

40. The array of claim 34, wherein the covalent interaction is a disulfide bond.

41. The array of claim 33, wherein the non-covalent interaction is an epitope-antibody interaction.

42. The array of claim 33, wherein the non-covalent interaction is a poly-histidine-metal cation interaction.

43. The array of claim 33, wherein the non-covalent interaction is nonspecific.

44. The array of claim 28, wherein the solid support is glass.

45. The array of claim 28, wherein the solid support is a polymer.

46. The array of claim 28, wherein the solid support comprises a metal surface.

47. The array of claim 28, wherein the solid support comprises a self-assembled monolayer.

48. A method of preparing an array of proteins, the method comprising the steps of:
providing a solid support, wherein the solid support is functionalized with a chemical moiety capable of interacting with a protein;
providing one or more solutions of one or more types of proteins to be attached to the solid support; and
delivering said one or more solutions to the solid support, whereby each of the proteins is attached to the solid support through an interaction, and whereby the array of proteins has a density of at least 1000 spots per cm².

49. The method of claim 48, wherein the solid support is glass.

50. The method of claim 48, wherein the solid support is a polymer.

51. The method of claim 48, wherein the solid support comprises a metal surface.

52. The method of claim 48, wherein the solid support comprises a self-assembled monolayer.

53. The method of claim 48, wherein the proteins attached to the array are functional.

54. The method of claim 48, wherein the linkage is a covalent interaction.

55. The method of claim 48, wherein the linkage is a non-covalent interaction.

56. The method of claim 48, wherein the density is at least 1500 spots per cm².

57. The method of claim 48, further comprising the step of blocking unoccupied sites on the solid support with a blocking agent.

58. The method of claim 57, wherein the blocking agent is selected from the group consisting of bovine serum albumin (BSA), caseine, nonfat milk, glycine, ethylenediamine, and ethanolamine.

59. The method of claim 48, wherein the solid support comprises a polymer functionalized with chemically activated groups.

60. The method of claim 48, wherein the solutions are aqueous solutions.

61. A method of preparing an array of proteins, the method comprising steps of:
providing a solid support, wherein the solid support is coated with bovine serum albumin (BSA) functionalized with a chemical moiety capable of interacting with a protein;
providing one or more solutions of one or more types of proteins to be attached to the solid support; and
delivering said one or more solutions to the solid support, whereby each of the proteins is attached to the solid support through a covalent interaction, and whereby the array of proteins has a density of at least 1000 spots per cm².

62. The method of claim 61, wherein the solid support is glass.

63. The method of claim 61, wherein the solid support is a polymer.

64. The method of claim 61, wherein the solid support comprises a metal surface.

65. The method of claim 61, wherein the solid support comprises a self-assembled monolayer.

66. The method of claim 61, wherein a substantial fraction of the proteins are functional.

67. The method of claim 61, wherein the interaction is a covalent interaction.

68. The method of claim 61, wherein the interaction is a non-covalent interaction.

69. The method of claim 61, wherein the density is at least 1500 spots per cm².

70. The method of claim 61, wherein the solutions are aqueous solutions.

71. A method of determining the interaction of protein with a molecule of interest, the method comprising the steps of:
providing an array of one or more types of proteins, wherein the array of proteins has a density of at least 1000 spots per cm²;
contacting the array with one or more types of molecules of interest; and
determining the interaction of specific protein-molecule partners.

72. The method of claim 71, wherein the molecule of interest is a biological macromolecule.

73. The method of claim 72, wherein the biological macromolecule is a protein.

74. The method of claim 73, wherein the biological macromolecule is a protein kinase.

75. The method of claim 73, wherein the biological macromolecule is an antibody.

76. The method of claim 72, wherein the biological macromolecule is a nucleic acid.

77. The method of claim 71, wherein the molecule of interest is a small molecule.

78. The method of claim 71, wherein the array comprises a solid support of glass.

79. The method of claim 71, wherein the array comprises an array of functional proteins.

80. The method of claim 71, wherein the array comprises an array of proteins covalently attached to a solid support.

81. The method of claim 71, wherein the interaction is an enzyme-substrate interaction.

82. A method of determining the interaction of protein with a molecule of interest, the method comprising the steps of:
providing an array of one or more types of proteins, wherein the proteims are attached to bovine serum albumin attached to a solid support, and wherein the array of proteins has a density of at least 1000 spots per cm²;
contacting the array with one or more types of molecules of interest; and
determining the interaction of specific protein-molecule partners.

83. The method of claim 82, wherein the molecule of interest is a biological macromolecule.

84. The method of claim 83, wherein the biological macromolecule is a protein.

85. The method of claim 84, wherein the biological macromolecule is a protein kinase.

86. The method of claim 84, wherein the biological macromolecule is an antibody.

87. The method of claim 83, wherein the biological macromolecule is a nucleic acid.

88. The method of claim 82, wherein the molecule of interest is a small molecule.

89. The method of claim 82, wherein the array comprises a solid support of glass.

90. The method of claim 82, wherein the array comprises an array of functional proteins.

91. The method of claim 82, wherein the array comprises an array of proteins covalently attached to BSA attached to a solid support.

92. The method of claim 82, wherein the interaction is an enzyme-substrate interaction.

93. A method for identifying molecules able to compete with a ligand for binding to a chemical compound, the method comprising steps of:
providing a solid support coated with a chemical compound;
contacting the solid support with a ligand known to bind to the chemical compound;
contacting the solid support with a molecule to be screened for its ability to compete with the known ligand for binding to the chemical compound; and
determining loss of the ligand known to bind to the chemical compound.

94. The method of claim 93 wherein the chemical compound is selected from the group consisting of proteins, peptides, polynucleotides, small molecules, carbohydrates, and lipids.

95. The method of claim 93 wherein the molecule to be screened is selected from the group consisting of peptides, proteins, small molecules, carbohydrates, lipids, and polynucleotides.

96. The method of claim 93 wherein the molecule to be screened is labeled with a fluorophore.

97. The method of claim 93 wherein the ligand is selected from the group consisting of peptides, proteins, small molecules, carbohydrates, lipids, and polynucleotides.

98. The method of claim 93 wherein the ligand is labeled with a fluorophore.

99. The method of claim 93 wherein the step of determining comprises detecting fluorescence of the labeled ligand.

100. The method of claim 93, wherein the chemical compound is a protein, and wherein the ligand is a peptide.

101. The method of claim 93, wherein the chemical compound is a protein, and wherein the ligand is a small molecule.

102. The method of claim 93, wherein the chemical compound is a protein receptor.

103. The method of claim 93, wherein the step of contacting with a ligand comprises arraying the ligand onto the coated solid support.

104. The method of claim 93, wherein the step of contacting the solid support with a molecule comprises arraying the molecule onto the coated solid support.
